(19) United States
(12) Patent Application Publication
(10) Pub. No.: US 2026/0209176 A1
(43) Pub. Date: Jul. 23, 2026

(54) CARBAZOLE-BASED BIS-CHALCONE, PREPARATION METHOD AND USE THEREOF

(71) Applicant: Guizhou Education University, Guiyang City (CN)

(72) Inventors: Hujun SHEN, Guiyang City (CN); Qingqing WU, Guiyang City (CN); Yi LIU, Guiyang City (CN)

(73) Assignee: Guizhou Education University, Guiyang City (CN)

(21) Appl. No.: 19/396,041

(22) Filed: Nov. 20, 2025

(30) Foreign Application Priority Data
Jan. 23, 2025 (CN) 202510105436.5

(51) Int. Cl. C07D 209/88; G03F 7/031

(52) U.S. Cl. CPC C07D 209/88; G03F 7/031

(57) ABSTRACT

Provided are a carbazole-based bis-chalcone, a preparation method and use thereof. The carbazole-based bis-chalcone has a chemical structural formula of Formula I, where in the Formula I, R is selected from the group consisting of —H, —SMe, —OMe, NMe₂, NEt₂, CF₃, and —N(Ph)₂.

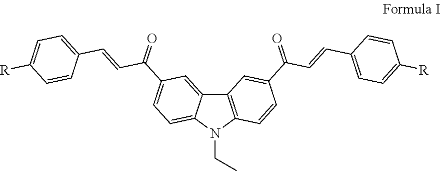

### CROSS REFERENCE TO RELATED APPLICATION

This patent application claims the benefit and priority of Chinese Patent Application No. 202510105436.5 filed with the China National Intellectual Property Administration on Jan. 23, 2025, the disclosure of which is incorporated by reference herein in its entirety as part of the present application.

### TECHNICAL FIELD

The present disclosure belongs to the technical field of photoinitiators, and in particular relates to a carbazole-based bis-chalcone, a preparation method and use thereof.

### BACKGROUND

Acrylate polymers have excellent weatherability, good adhesion and optical properties, and good flexibility and elasticity, and are widely used in many fields. Acrylate polymers are primarily formed by using photoinitiators, which generate active radicals under light exposure to initiate the polymerization reaction of monomers. Deep curing refers to the process during which a material undergoes sufficient polymerization reaction from the surface to the interior, forming a highly cross-linked three-dimensional network structure. Deep-cured materials exhibit better properties. For example, a deep-cured coating offers enhanced wear resistance, corrosion resistance, and chemical resistance.

Photoinitiators are a critical component in photocuring systems, whose performance directly influences the efficiency of the curing process, the degree of curing, and the properties of the cured product. Current commonly used photoinitiators, such as camphorquinone (CQ) and 2-isopropylthioxanthone (ITX), require being combined with a hydrogen donor to achieve high initiation activity, along with issues such as migration and the fact that ITX has reproductive toxicity, significantly limiting their application in biopharmaceuticals and other fields. The development of single-component initiators and low-toxicity photoinitiators has consistently remained a research focus in the field of photopolymerization.

### SUMMARY

An object of the present disclosure is to provide a carbazole-based bis-chalcone, a preparation method and use thereof. In the present disclosure, the carbazole-based bis-chalcone, when used as a photoinitiator to initiate a polymerization reaction of acrylate monomers, shows a rapid photoinitiation rate and a high double-bond conversion and can be used in deep-curing systems.

In order to achieve the above object, the present disclosure provides the following technical solutions:

The present disclosure provides a carbazole-based bis-chalcone having a chemical structural formula of Formula I:

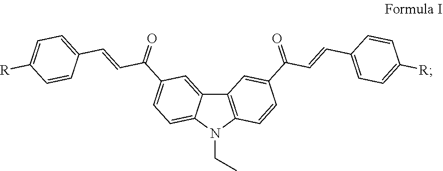

where in the Formula I, R is selected from the group consisting of —H, —SMe, —OMe, NMe₂, NEt₂, CF₃, and —N(Ph)₂; and Me represents methyl, Et represents ethyl, and Ph represents phenyl.

The present disclosure further provides a method for preparing the carbazole-based bis-chalcone as described in the aforementioned technical solutions, the method including: mixing 3,6-diacetyl-N-ethylcarbazole, an alkali, a solvent, and benzaldehyde/(or) benzaldehyde derivative, and subjecting a resulting mixture to a Claisen-Schmidt reaction to obtain the carbazole-based bis-chalcone.

In some embodiments, a molar ratio of the benzaldehyde/benzaldehyde derivative to the 3,6-diacetyl-N-ethylcarbazole is in a range of 2:1 to 3:1.

In some embodiments, a ratio of an amount of substance of the 3,6-diacetyl-N-ethylcarbazole to a volume of the solvent is in a range of 5 mmol:30-80 mL.

In some embodiments, the Claisen-Schmidt reaction is conducted at a temperature of 30° C. to 80° C. for 8 h to 15 h.

The present disclosure further provides use of the carbazole-based bis-chalcone as described in the aforementioned technical solutions or the carbazole-based bis-chalcone prepared by the method as described in the aforementioned technical solutions as a photoinitiator.

In some embodiments, the carbazole-based bis-chalcone is used as the photoinitiator to initiate a polymerization reaction of an acrylate monomer.

In some embodiments, the carbazole-based bis-chalcone is used in combination with ethyl p-dimethylaminobenzoate or an iodonium salt as the photoinitiator to initiate a polymerization reaction of an acrylate monomer.

In some embodiments, the acrylate monomer includes at least one selected from the group consisting of methyl methacrylate, butyl methacrylate, methyl acrylate, n-butyl acrylate, 1,6-hexanediol diacrylate, trimethylolpropane triacrylate, pentaerythritol triacrylate, 2-hydroxypropyl methacrylate or 2-hydroxyethyl acrylate, 2,2-bis[4-(2-hydroxy-3-methacryloyloxypropoxy)phenyl]propane, triethylene glycol dimethacrylate, 3,3,4,4,5,5,6,6,7,7,8,8,8-tridecafluorooctyl acrylate, 2,2,3,3,4,4,5,5,6,6,7,7,8,8,8-pentadecafluorooctyl acrylate, hexafluorobutyl acrylate, pentafluorophenyl methacrylate, 2,2,2-trifluoroethyl acrylate, 2-(perfluorooctyl)ethyl methacrylate, 2-(perfluorobutyl)ethyl methacrylate, 2-(perfluorohexyl)ethyl acrylate, 2-(perfluorohexyl)ethyl methacrylate, 2,2,3,3-tetrafluoropropyl methacrylate, 2,2,3,3,3-pentafluoropropyl acrylate, 1H,1H,2H,2H-perfluorodecyl acrylate, 1H,2H,3H,4H-perfluorooctanol acrylate, 2,2,3,3,4,4,4-heptafluorobutyl acrylate, 2,2,3,3,4,4,4-heptafluorobutyl methacrylate, 2,2,3,4,4,4-hexafluorobutyl methacrylate, 1,1,1,3,3,3-hexafluoropropan-2-yl acrylate, and 2,2,3,3,4,4,5,5-octafluoro-1,6-hexanediol diacrylate.

In some embodiments, a light source for the polymerization reaction is an LED (light-emitting diode) light source.

The present disclosure provides a carbazole-based bis-chalcone having a chemical structural formula of Formula I:

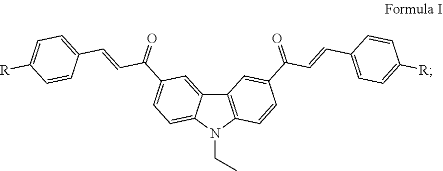

where in Formula I, R is selected from the group consisting of —H, —SMe, —OMe, NMe₂, NEt₂, CF₃, and —N(Ph)₂. In the present disclosure, the specific structure of the carbazole-based bis-chalcone is controlled such that the carbazole-based bis-chalcone, when used as a photoinitiator to initiate a polymerization reaction of acrylate monomers, shows a rapid photoinitiation rate and a high double-bond conversion, and can be used in deep-curing systems.

### BRIEF DESCRIPTION OF THE DRAWINGS

FIG. 1 shows the reaction equation for preparing the carbazole-based bis-chalcone of an embodiment of the present disclosure;

FIG. 2 shows ¹H nuclear magnetic resonance (¹H NMR) spectra of the carbazole-based bis-chalcone prepared in Example 1;

FIG. 3 shows ¹³C nuclear magnetic resonance (¹³C NMR) spectra of the carbazole-based bis-chalcone prepared in Example 1;

FIG. 4 shows ¹H NMR spectra of the carbazole-based bis-chalcone prepared in Example 2;

FIG. 5 shows ¹³C NMR spectra of the carbazole-based bis-chalcone prepared in Example 2;

FIG. 6 shows ¹H NMR spectra of the carbazole-based bis-chalcone prepared in Example 3;

FIG. 7 shows ¹³C NMR spectra of the carbazole-based bis-chalcone prepared in Example 3;

FIG. 8 shows carbon-carbon double bond conversion-time profiles of the 1,6-hexanediol diacrylate monomer in Use Examples 1-3;

FIG. 9 shows carbon-carbon double bond conversion-time profiles of the 1,6-hexanediol diacrylate monomer in Use Examples 4-6;

FIG. 10 shows carbon-carbon double bond conversion-time profiles of the 1,6-hexanediol diacrylate monomer in Use Examples 7-9;

FIG. 11 shows carbon-carbon double bond conversion-time profiles of the 1,6-hexanediol diacrylate monomer in Use Examples 3, 6, and 9 and Comparative Use Examples 1-3;

FIG. 12 shows a macrograph of the deep-cured polymer obtained in Use Example 10; and

FIG. 13 shows a macrograph of the deep-cured polymer obtained in Use Example 11.

### DETAILED DESCRIPTION OF THE EMBODIMENTS

The present disclosure provides a carbazole-based bis-chalcone having a chemical structural formula of Formula I.

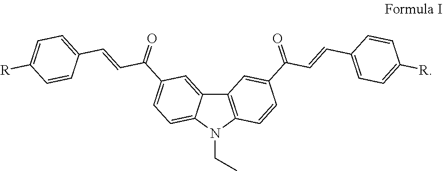

In the present disclosure, in the formula I, R is selected from the group consisting of —H, —SMe, —OMe, NMe₂, NEt₂, CF₃, and —N(Ph)₂.

As an embodiment, under the condition that R is —N(Ph)₂, the carbazole-based bis-chalcone has a chemical structural formula of

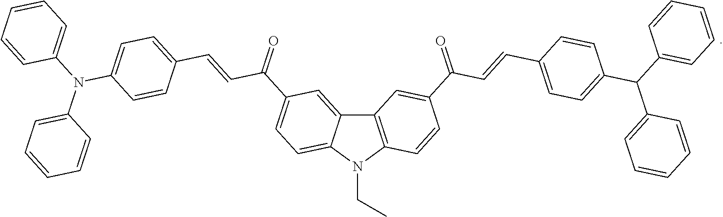 [SMILES: CCn1c2ccc(C(=O)/C=C/c3ccc(C(c4ccccc4)c4ccccc4)cc3)cc2c2cc(C(=O)/C=C/c3ccc(N(c4ccccc4)c4ccccc4)cc3)ccc21]

In the present disclosure, the carbazole-based bis-chalcone as an LED photoinitiator can act as a single-component photoinitiator to initiate the polymerization of (meth)acrylate monomers and acrylate monomers or methacrylate monomers grafted with a fluorine-containing segment, or can be used in combination with a hydrogen-donor amine (ethyl p-dimethylaminobenzoate) or an iodonium salt as a photoinitiator; and when used in combination, the carbazole-based bis-chalcone has a higher photoinitiation activity and can be used in deep-curing systems, allowing for the preparation of thicker polymer materials under light exposure. In the present disclosure, the carbazole-based bis-chalcone does not require the addition of a solvent for dissolution, nor does it require an auxiliary agent to effectively initiate monomer polymerization, and has a higher initiation activity than existing photoinitiators.

The present disclosure further provides a method for preparing the carbazole-based bis-chalcone as described in the aforementioned technical solutions, the method including:
mixing 3,6-diacetyl-N-ethylcarbazole (3,6-diacetyl-9-ethyl-9H-carbazole), an alkali, a solvent, and benzaldehyde/benzaldehyde derivative, and subjecting a resulting mixture to a Claisen-Schmidt reaction to obtain the carbazole-based bis-chalcone.

In the present disclosure, unless otherwise specified, there is no particular limitation on the source of the raw materials, and commercially-available products well known to those skilled in the art may be used.

In some embodiments of the present disclosure, the 3,6-diacetyl-N-ethylcarbazole has a structural formula of

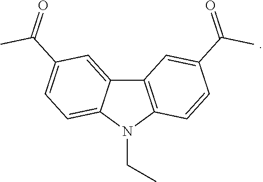 [SMILES: CCn1c2ccc(C(C)=O)cc2c2cc(C(C)=O)ccc21]

In some embodiments of the present disclosure, the benzaldehyde/benzaldehyde derivative has a structural formula of

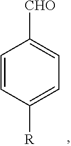

where R is selected from the group consisting of —H, —SMe, —OMe, NMe₂, NEt₂, CF₃, and —N(Ph)₂.

In some embodiments of the present disclosure, a molar ratio of the benzaldehyde/benzaldehyde derivative to the 3,6-diacetyl-N-ethylcarbazole is in a range of 2:1 to 3:1. As an embodiment, the molar ratio of the benzaldehyde/benzaldehyde derivative to the 3,6-diacetyl-N-ethylcarbazole is 2:1, 2.1:1, 2.2:1, 2.3:1, 2.4:1, 2.5:1, 2.6:1, 2.7:1, 2.8:1, 2.9:1 or 3:1. In the present disclosure, controlling the molar ratio of the benzaldehyde/benzaldehyde derivative to the 3,6-diacetyl-N-ethylcarbazole within the ranges above ensures a complete reaction between the two substances, resulting in a higher yield.

In some embodiments of the present disclosure, the alkali includes potassium hydroxide or sodium hydroxide. In the present disclosure, the alkali acts as a catalyst.

In some embodiments of the present disclosure, the solvent includes at least one of water and ethanol.

In some embodiments of the present disclosure, a ratio of an amount of substance of the 3,6-diacetyl-N-ethylcarbazole to a volume of the solvent is in a range of 5 mmol:30-80 mL. As an embodiment, the ratio of the amount of substance of the 3,6-diacetyl-N-ethylcarbazole to the volume of the solvent is 5 mmol:30 mL, 5 mmol:40 mL, 5 mmol:50 mL, 5 mmol:60 mL, 5 mmol:70 mL, or 5 mmol:80 mL. In the present disclosure, controlling the ratio of the amount of substance of 3,6-diacetyl-N-ethylcarbazole to the volume of the solvent within the ranges above enables the full dissolution of the raw materials.

In some embodiments of the present disclosure, the mixing of the 3,6-diacetyl-N-ethylcarbazole, the benzaldehyde/benzaldehyde derivative, the alkali, and the solvent is conducted by mixing the alkali and the solvent to obtain an alkaline solution, and adding the 3,6-diacetyl-N-ethylcarbazole and the benzaldehyde/benzaldehyde derivative in sequence.

In some embodiments of the present disclosure, a mass concentration of the alkaline solution is in a range of 10% to 30%. As an embodiment, the mass concentration of the alkaline solution is 10%, 15%, 20%, 25% or 30%. In the present disclosure, there is no special limitation on the amount of the alkali, as long as the mass concentration of the alkaline solution could fall within the ranges above. In the present disclosure, controlling the mass concentration of the alkaline solution within the ranges above ensures the Claisen-Schmidt reaction proceeds completely.

In some embodiments of the present disclosure, the Claisen-Schmidt reaction is conducted at a temperature of 30° C. to 80° C. As an embodiment, the Claisen-Schmidt reaction is conducted at 30° C., 40° C., 50° C., 60° C., 70° C. or 80° C.

In some embodiments of the present disclosure, the Claisen-Schmidt reaction is conducted for 8 h to 15 h. As an embodiment, the Claisen-Schmidt reaction is conducted for 8 h, 9 h, 10 h, 11 h, 12 h, 13 h, 14 h, or 15 h. In the present disclosure, controlling the temperature and duration of the Claisen-Schmidt reaction within the ranges above ensures the reaction proceeds completely, thereby increasing the reaction yield.

In some embodiments of the present disclosure, the method further includes after the Claisen-Schmidt reaction is completed, subjecting a product of the Claisen-Schmidt reaction to cooling, filtration, washing, and drying in sequence to obtain the carbazole-based bis-chalcone.

In some embodiments of the present disclosure, the cooling is conducted in ice water. In the present disclosure, there is no special limitation on the cooling time, as long as the product could be cooled to room temperature.

In the present disclosure, there is no special limitation on the filtration operation, and any technical solution of filtration that is well known to those skilled in the art for soil-liquid separation may be used.

In the present disclosure, there is no special limitation on the washing operation, and any technical solution of washing that is well known to those skilled in the art for removing unreacted raw materials, etc may be used. In an embodiment of the present disclosure, the washing is conducted 2 times to 3 times with ethanol, with 10 mL of ethanol for each washing.

In the present disclosure, there is no special limitation on the drying operation, any technique solution of drying that is well-known to those skilled in the art for drying to a constant weight may be used.

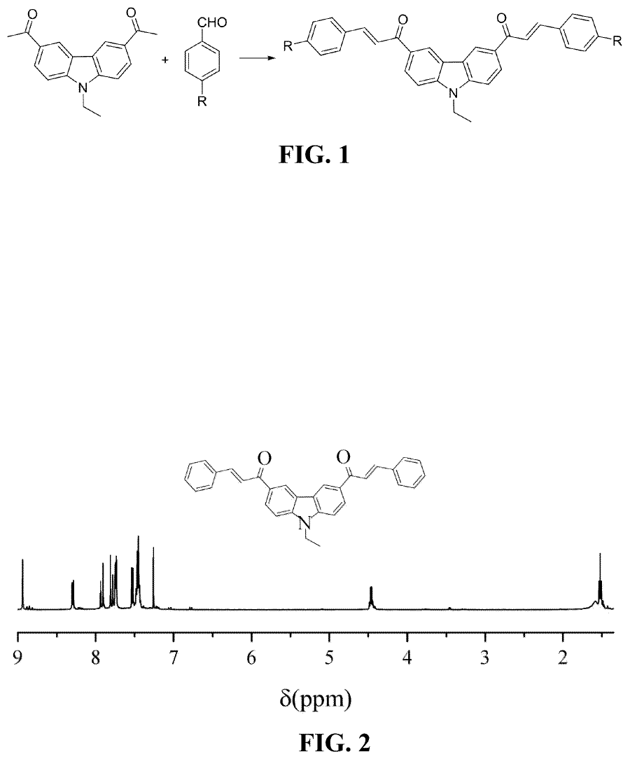

The reaction equation for preparing the carbazole-based bis-chalcone according to an embodiment of the present disclosure is shown in FIG. 1.

The present disclosure further provides use of the carbazole-based bis-chalcone as described in the aforementioned technical solutions or the carbazole-based bis-chalcone prepared by the method as described in the aforementioned technical solutions as a photoinitiator.

In some embodiments of the present disclosure, the carbazole-based bis-chalcone is used as the photoinitiator to initiate a polymerization reaction of an acrylate monomer.

In some embodiments of the present disclosure, the carbazole-based bis-chalcone is used in combination with ethyl p-dimethylaminobenzoate or an iodonium salt as the photoinitiator to initiate a polymerization reaction of an acrylate monomer.

In some embodiments of the present disclosure, the iodonium salt is selected from the group consisting of diphenyliodonium hexafluorophosphate, bis(tert-butylphenyl)iodonium hexafluorophosphate, and bis(p-tolyl)iodonium hexafluorophosphate, more preferably bis(tert-butylphenyl)iodonium hexafluorophosphate. In the present disclosure, when the carbazole-based bis-chalcone is used in combination with ethyl p-dimethylaminobenzoate or an iodonium salt as a photoinitiator to initiate a polymerization reaction of acrylate monomers, the carbazole-based bis-chalcone has a higher initiation activity.

In some embodiments of the present disclosure, a molar ratio of the carbazole-based bis-chalcone to the ethyl p-dimethylaminobenzoate or iodonium salt is in a range of 1:1 to 1:2. In the present disclosure, controlling the molar ratio of the carbazole-based bis-chalcone to the ethyl p-dimethylaminobenzoate or iodonium salt within the ranges above can ensure a higher initiation activity.

In some embodiments of the present disclosure, the acrylate monomer includes at least one selected from the group consisting of methyl methacrylate, butyl methacrylate, methyl acrylate, n-butyl acrylate, 1,6-hexanediol diacrylate, trimethylolpropane triacrylate, pentaerythritol triacrylate, 2-hydroxypropyl methacrylate or 2-hydroxyethyl acrylate, 2,2-bis[4-(2-hydroxy-3-methacryloyloxypropoxy)phenyl]propane, triethylene glycol dimethacrylate, 3,3,4,4,5,5,6,6,7,7,8,8,8-tridecafluorooctyl acrylate, 2,2,3,3,4,4,5,5,6,6,7,7,8,8,8-pentadecafluorooctyl acrylate, hexafluorobutyl acrylate, pentafluorophenyl methacrylate, 2,2,2-trifluoroethyl acrylate, 2-(perfluorooctyl)ethyl methacrylate, 2-(perfluorobutyl)ethyl methacrylate, 2-(perfluorohexyl)ethyl acrylate, 2-(perfluorohexyl)ethyl methacrylate, 2,2,3,3-tetrafluoropropyl methacrylate, 2,2,3,3,3-pentafluoropropyl acrylate, 1H,1H,2H,2H-perfluorodecyl acrylate, 1H,2H,3H,4H-perfluorooctanol acrylate, 2,2,3,3,4,4,4-heptafluorobutyl acrylate, 2,2,3,3,4,4,4-heptafluorobutyl methacrylate, 2,2,3,4,4,4-hexafluorobutyl methacrylate, 1,1,1,3,3,3-hexafluoropropan-2-yl acrylate, and 2,2,3,3,4,4,5,5-octafluoro-1,6-hexanediol diacrylate. In the present disclosure, the carbazole-based bis-chalcone as a photoinitiator, can initiate a polymerization reaction of a variety of monomers, including fluoromonomers, enabling it to have a wider application potential in the development and application of a variety of polymer materials, including fluoropolymer materials.

In some embodiments of the present disclosure, a ratio of an amount of substance of the carbazole-based bis-chalcone to a mass of the acrylate monomer is in a range of 0.025 mmol:5-15 g, more preferably 0.025 mmol:10 g. In the present disclosure, controlling the ratio of the amount of substance of the carbazole-based bis-chalcone to the mass of the acrylate monomer within the ranges above can ensure a higher initiation activity.

In some embodiments of the present disclosure, a light source for the polymerization reaction is an LED light source. In some embodiments of the present disclosure, the LED light source has a wavelength of 365 nm to 405 nm; the light source has an irradiance of 100 mW/cm² to 200 mW/cm², more preferably 130 mW/cm² to 200 mW/cm². In some embodiments of the present disclosure, the polymerization reaction is conducted for 1 min to 30 min. In the present disclosure, the use of the light source with wavelengths above can result in better initiation activity.

In the present disclosure, the carbazole-based bis-chalcone as a photoinitiator, offers a rapid photoinitiation rate and a high double-bond conversion and can be used in deep-curing systems, allowing for the preparation of thicker polymers.

The technical solutions in the present disclosure will be described clearly and completely below with reference to the examples of the present disclosure. Apparently, the described examples are merely some, rather than all of the examples of the present disclosure. Based on the examples in the present disclosure, all other examples obtained by those of ordinary skill in the art without involving inventive effort belong to the scope of the present disclosure.

### Example 1

A carbazole-based bis-chalcone had the following chemical structural formula:

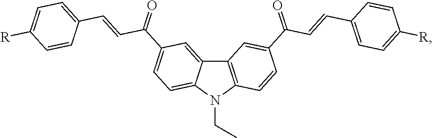

where R was —H.

The carbazole-based bis-chalcone was prepared by using a method as follows: potassium hydroxide and water were mixed to obtain an aqueous potassium hydroxide solution with a mass concentration of 20%, 50 mL of the aqueous potassium hydroxide solution with the mass concentration of 20%, 3,6-diacetyl-N-ethylcarbazole (1.395 g, 5 mmol), and benzaldehyde (1.272 g, 12 mmol) were added in sequence to a 250 mL three-neck flask, and heated to 50° C. and subjected to a reaction for 12 h. After the reaction was completed, a resulting system was cooled in ice water, and subjected to filtration to obtain a crude product. The crude product was washed twice with ethanol (each 10 mL) and dried to obtain the carbazole-based bis-chalcone as a yellow solid (1.85 g, yield 81.3%), which was designated as DCZ.

The ¹H NMR spectra of the carbazole-based bis-chalcone prepared in Example 1 is shown in FIG. 2. ¹H NMR spectra data: ¹H NMR (CDCl₃, 500 MHz) δ ppm: 1.49-1.55 (t, J=7.0 Hz, 3H, CH₂CH₃), 4.42-4.50 (q, J=7.0 Hz, 2H, CH₂CH₃), 7.43-7.50 (m, 4H), 7.51-7.55 (d, J=8.5 Hz, 2H), 7.69-7.76 (d, J=7.5 Hz, 4H), 7.76-7.83 (m, 2H), 7.88-7.96 (m, 2H), 8.27-8.32 (dd, J₁=8.5 Hz, J₂=1.5 Hz, 2H), 8.94 (s, 2H).

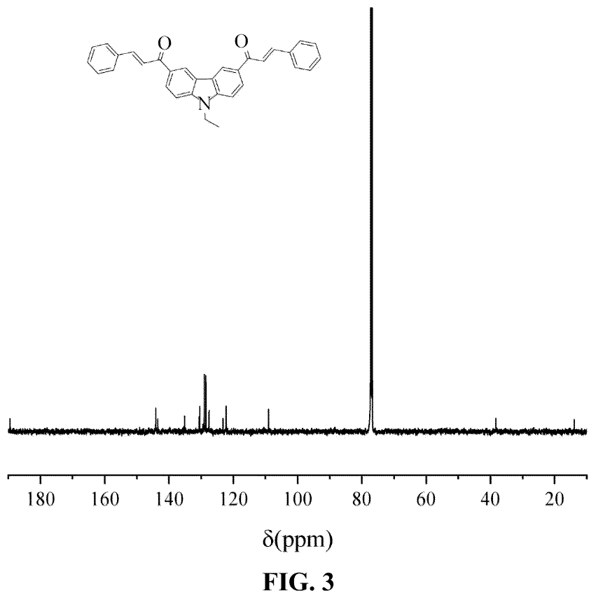

The ¹³C NMR spectra of the carbazole-based bis-chalcone prepared in Example 1 is shown in FIG. 3. ¹³C NMR spectra data: ¹³C NMR (CDCl₃, 125 MHz) δ ppm: 189.39 (C═O), 144.15, 143.47, 135.21, 130.67, 130.40, 129.00, 128.51, 127.51, 123.20, 122.27, 122.18, 109.01, 38.28 (NCH₂), 13.94 (CH₃).

### Example 2

A carbazole-based bis-chalcone had the following chemical structural formula:

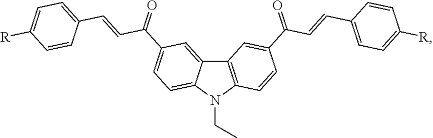

where R was —SMe.

The carbazole-based bis-chalcone was prepared by using a method as follows: potassium hydroxide and water were mixed to obtain an aqueous potassium hydroxide solution with a mass concentration of 20%, 50 mL of the aqueous potassium hydroxide solution with the mass concentration of 20%, 3,6-diacetyl-N-ethylcarbazole (1.395 g, 5 mmol), and 4-(methylthio)benzaldehyde (1.824 g, 12 mmol) were added in sequence to a 250 mL three-neck flask, and heated to 60° C. and subjected to a reaction for 10 h. After the reaction was completed, a resulting system was cooled in ice water, and subjected to filtration to obtain a crude product. The crude product was washed three times with ethanol (each 10 mL) and dried to obtain the carbazole-based bis-chalcone as a yellow solid (2.31 g, yield 84.6%), which was designated as DCZ-SMe.

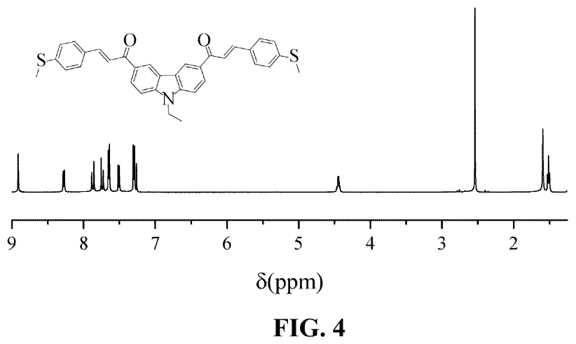

The ¹H NMR spectra of the carbazole-based bis-chalcone prepared in Example 2 is shown in FIG. 4. ¹H NMR spectra data: ¹H NMR (CDCl₃, 500 MHz) δ ppm: 1.44-1.56 (t, J=7.0 Hz, 3H, CH₂CH₃), 2.54 (s, 6H, SCH₃), 4.38-4.49 (q, J=7.0 Hz, 2H, CH₂CH₃), 7.27-7.36 (d, J=8.5 Hz, 4H), 7.47-7.55 (d, J=8.5 Hz, 2H), 7.60-7.67 (d, J=8.0 Hz, 4H), 7.71-7.78 (m, 2H), 7.82-7.91 (m, 2H), 8.23-8.31 (dd, J₁=8.5 Hz, J₂=1.5 Hz, 2H), 8.91 (s, 2H).

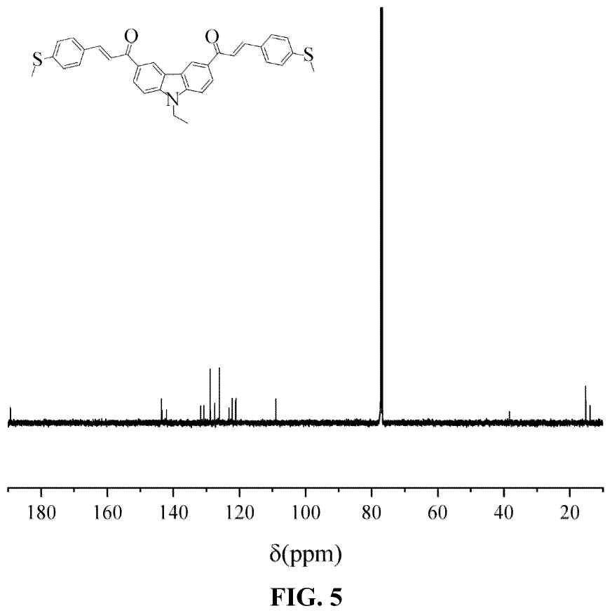

The ¹³C NMR spectra of the carbazole-based bis-chalcone prepared in Example 2 is shown in FIG. 5. ¹³C NMR spectra data: ¹³C NMR (CDCl₃, 125 MHz) δ ppm: 189.28 (C═O), 143.62, 143.42, 142.12, 131.72, 130.74, 128.88, 127.46, 126.09, 123.19, 122.19, 121.07, 108.98, 38.27 (NCH₂), 15.23 (SCH₃), 13.93 (CH₂CH₃).

### Example 3

A carbazole-based bis-chalcone had the following chemical structural formula:

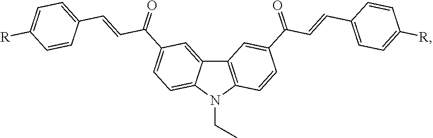

where R was —N(Ph)₂.

The carbazole-based bis-chalcone was prepared by using a method as follows: potassium hydroxide and water were mixed to obtain an aqueous potassium hydroxide solution with a mass concentration of 20%, 50 mL of the aqueous potassium hydroxide solution with the mass concentration of 20%, 3,6-diacetyl-N-ethylcarbazole (1.395 g, 5 mmol), and 4-(diphenylamino)benzaldehyde (3.280 g, 12 mmol) were added in sequence to a 250 mL three-neck flask, and heated to 60° C. and subjected to a reaction for 10 h. After the reaction was completed, a resulting system was cooled in ice water, and subjected to filtration to obtain a crude product. The crude product was washed three times with ethanol (each 10 mL) and dried to obtain the carbazole-based bis-chalcone as a yellow solid (3.27 g, yield 82.8%), which was designated as DCZ-DB.

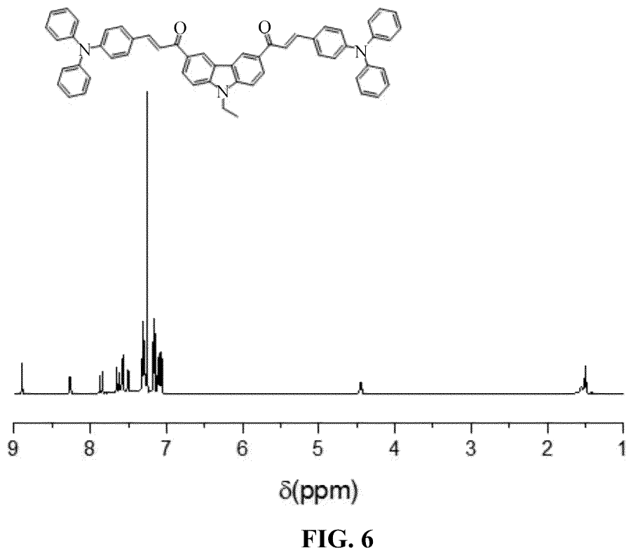

The ¹H NMR spectra of the carbazole-based bis-chalcone prepared in Example 3 is shown in FIG. 6. ¹H NMR spectra data: ¹H NMR (500 MHz, CDCl₃) 1.48-1.53 (3H, CH₃, t, J=7.0 Hz), 4.41-4.48 (2H, CH₂, q, J=7.0 Hz), 7.05-7.12 (8H, q, J=8.5 Hz), 7.14-7.18 (8H, d, J=8.0 Hz), 7.27-7.33 (8H, t, J=7.0 Hz), 7.49-7.62 (2H, d, J=8.5 Hz), 7.55-7.59 (4H, d, J=8.5 Hz), 7.61-7.66 (2H, d, J=15.5 Hz), 7.83-7.88 (2H, d, J=15.5 Hz), 8.25-8.28 (2H, dd, J=1.5 Hz, J=14 Hz), 8.90 (2H, s).

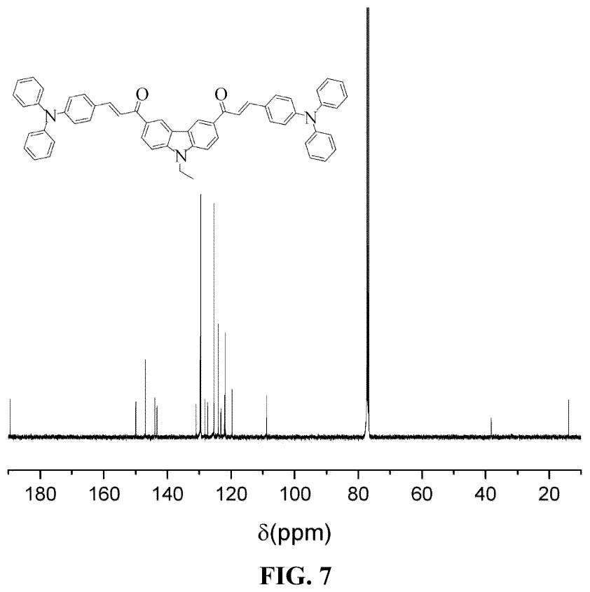

The ¹³C NMR spectra of the carbazole-based bis-chalcone prepared in Example 3 is shown in FIG. 7. ¹³C NMR spectra data: ¹³C NMR (125 MHz, CDCl₃) 189.50, 150.00, 146.93, 143.95, 143.27, 130.99, 129.73, 129.50, 128.26, 127.36, 125.40, 124.03, 123.17, 122.08, 121.80, 119.66, 108.85, 38.22, 13.91.

### Use Example 1

The carbazole-based bis-chalcone (2.5×10⁻⁵ mol, 0.0114 g) prepared in Example 1, and 10.00 g of 1,6-hexanediol diacrylate (HDDA) were weighed by using an analytical balance with a readability of 0.1 mg and placed in a 10 mL centrifuge tube, ultrasonically mixed until uniform, and exposed to an LED light source with a wavelength of 385 nm and an irradiance of 130 mW/cm² for photocuring to obtain a polymer.

### Use Example 2

The carbazole-based bis-chalcone prepared in Example 1 in Use Example 1 was replaced with 0.0137 g of carbazole-based bis-chalcone prepared in Example 2, and the other parameters were the same as those in Use Example 1.

### Use Example 3

The carbazole-based bis-chalcone prepared in Example 1 in Use Example 1 was replaced with 0.0197 g of carbazole-based bis-chalcone prepared in Example 3, and the other parameters were the same as those in Use Example 1.

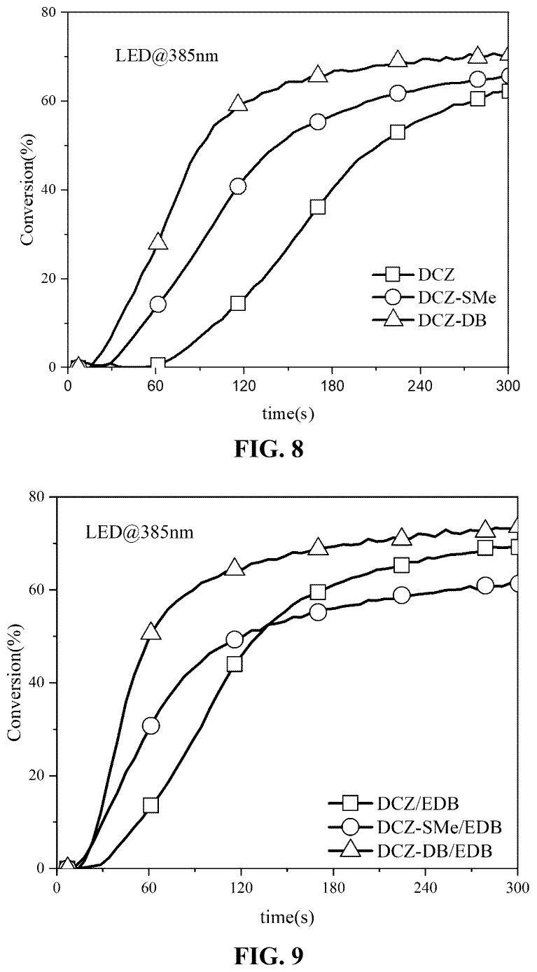

The photocuring kinetics in Use Examples 1-3 were monitored by real-time infrared spectroscopy. The resulting carbon-carbon double bond conversion-time profiles of the 1,6-hexanediol diacrylate monomer are shown in FIG. 8. As can be seen from FIG. 8, when used as a photoinitiator to initiate the photocuring of HDDA, the carbazole-based bis-chalcones of Examples 1-3 all result in rapid photoinitiation rates, with the carbazole-based bis-chalcone of Example 3 having the fastest photoinitiation activity and a high double bond conversion.

### Use Example 4

The carbazole-based bis-chalcone (2.5×10⁻⁵ mol, 0.0114 g) prepared in Example 1, ethyl 4-dimethylaminobenzoate (EDB) (0.0097 g, 5×10⁻⁵ mol), and 10.00 g of 1,6-hexanediol diacrylate (HDDA) were weighed by using an analytical balance with a readability of 0.1 mg and placed in a 10 mL centrifuge tube, ultrasonically mixed until uniform, and exposed to an LED light source with a wavelength of 385 nm and an irradiance of 130 mW/cm² for photocuring to obtain a polymer.

### Use Example 5

The carbazole-based bis-chalcone prepared in Example 1 in Use Example 4 was replaced with 0.0137 g of carbazole-based bis-chalcone prepared in Example 2, and the other parameters were the same as those in Use Example 4.

### Use Example 6

The carbazole-based bis-chalcone prepared in Example 1 in Use Example 4 was replaced with 0.0197 g of carbazole-based bis-chalcone prepared in Example 3, and the other parameters were the same as those in Use Example 4.

### Use Example 7

The carbazole-based bis-chalcone (2.5×10⁻⁵ mol, 0.0114 g) prepared in Example 1, bis(tert-butylphenyl)iodonium hexafluorophosphate (Iod) (0.0134 g, 2.5×10⁻⁵ mol), and 10.00 g of 1,6-hexanediol diacrylate (HDDA) were weighed by using an analytical balance with a readability of 0.1 mg and placed in a 10 mL centrifuge tube, ultrasonically mixed until uniform, and exposed to an LED light source with a wavelength of 385 nm and an irradiance of 130 mW/cm² for photocuring to obtain a polymer.

### Use Example 8

The carbazole-based bis-chalcone prepared in Example 1 in Use Example 7 was replaced with 0.0137 g of carbazole-based bis-chalcone prepared in Example 2, and the other parameters were the same as those in Use Example 7.

### Use Example 9

The carbazole-based bis-chalcone prepared in Example 1 in Use Example 7 was replaced with 0.0197 g of carbazole-based bis-chalcone prepared in Example 3, and the other parameters were the same as those in Use Example 7.

The photocuring kinetics in Use Examples 4-6 were monitored by real-time infrared spectroscopy. The resulting carbon-carbon double bond conversion-time profiles of the 1,6-hexanediol diacrylate monomer are shown in FIG. 9.

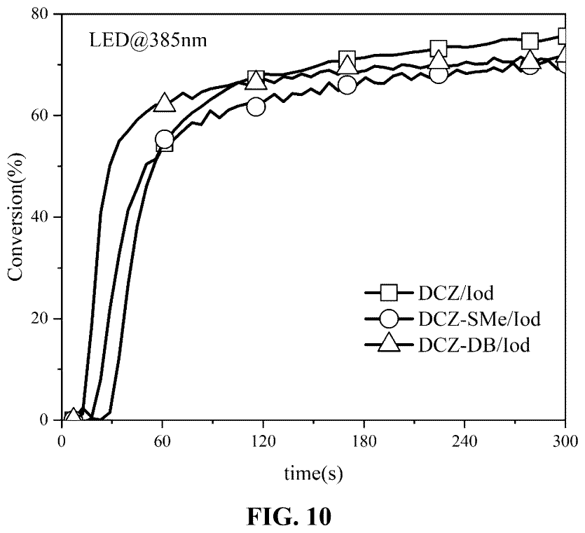

The photocuring kinetics in Use Examples 7-9 were monitored by real-time infrared spectroscopy. The resulting carbon-carbon double bond conversion-time profiles of the 1,6-hexanediol diacrylate monomer are shown in FIG. 10.

As can be seen from FIGS. 8-10, the carbazole-based bis-chalcone can be used as a single-component photoinitiator to initiate the polymerization of 1,6-hexanediol diacrylate, and can also be used in combination with ethyl 4-dimethylaminobenzoate or bis(tert-butylphenyl)iodonium hexafluorophosphate as a photoinitiator to initiate the polymerization of 1,6-hexanediol diacrylate, and achieves the best performance when used in combination with bis(tert-butylphenyl)iodonium hexafluorophosphate.

### Comparative Use Example 1

The carbazole-based bis-chalcone prepared in Example 3 in Use Example 3 was replaced with 0.0042 g of camphorquinone (CQ) as a photoinitiator, and the other parameters were the same as those in Use Example 3.

### Comparative Use Example 2

The carbazole-based bis-chalcone prepared in Example 3 in Use Example 6 was replaced with 0.0042 g of camphorquinone (CQ) as a photoinitiator, and the other parameters were the same as those in Use Example 6.

### Comparative Use Example 3

The carbazole-based bis-chalcone prepared in Example 3 in Use Example 9 was replaced with 0.0042 g of camphorquinone (CQ) as a photoinitiator, and the other parameters were the same as those in Use Example 9.

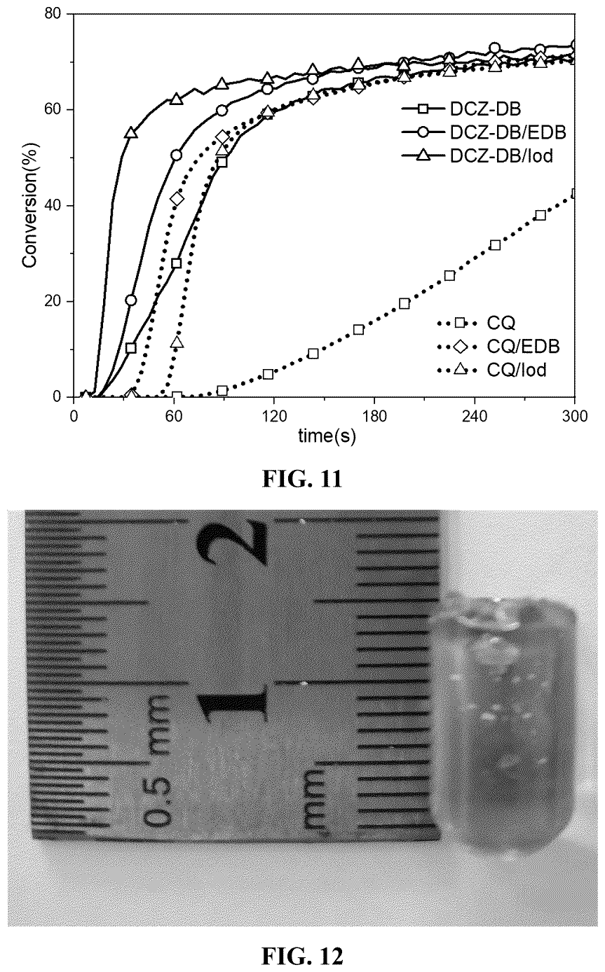

The photocuring kinetics in Application Examples 3, 6, and 9 and Comparative Use Examples 1-3 were monitored by real-time infrared spectroscopy. The resulting carbon-carbon double bond conversion-time profiles of the 1,6-hexanediol diacrylate monomer are shown in FIG. 11. As can be seen from FIG. 11, the carbazole-based bis-chalcone prepared in Example 3 has a better photoinitiation rate and a higher double bond conversion compared with the photoinitiator camphorquinone (CQ), with a more significant effect being observed especially in the single-component photoinitiation system.

### Use Example 10

The carbazole-based bis-chalcone (2.5×10⁻⁵ mol, 0.0197 g) prepared in Example 3, and 10.00 g of 1,6-hexanediol diacrylate (HDDA) were weighed by using an analytical balance with a readability of 0.1 mg and placed in a 10 mL centrifuge tube, ultrasonically mixed until uniform, and transferred to a flat-mouth test tube (with a diameter of 10 mm and a depth of 100 mm). Nitrogen was introduced for 2 min, and the tube was sealed with a sealing film and exposed to an LED light source with a wavelength of 385 nm and an irradiance of 200 mW/cm² for photocuring for 30 min. A resulting product was washed with ethanol to remove uncured samples to obtain a deep-cured polymer.

The macrograph of the deep-cured polymer prepared in Use Example 10 is shown in FIG. 12. As can be seen from FIG. 12, when the photocuring time is 30 min, a polymer curing depth of 1.5 cm is observed. It is thus demonstrated that the carbazole-based bis-chalcone can be used as a photoinitiator for the preparation of deep-cured materials, with a wider application range.

### Use Example 11

The carbazole-based bis-chalcone (2.5×10⁻⁵ mol, 0.0197 g) prepared in Example 3, bis(tert-butylphenyl)iodonium hexafluorophosphate (Iod) (0.0134 g, 2.5×10⁻⁵ mol), and 10.00 g of 1,6-hexanediol diacrylate (HDDA) were weighed by using an analytical balance with a readability of 0.1 mg and placed in a 10 mL centrifuge tube, ultrasonically mixed until uniform, and transferred to a flat-mouth test tube (with a diameter of 10 mm and a depth of 100 mm). Nitrogen was introduced for 2 min, and the tube was sealed with a sealing film and exposed to an LED light source with a wavelength of 385 nm and an irradiance of 200 mW/cm² for photocuring for 30 min. A resulting product was washed with ethanol to remove uncured samples to obtain a deep-cured polymer.

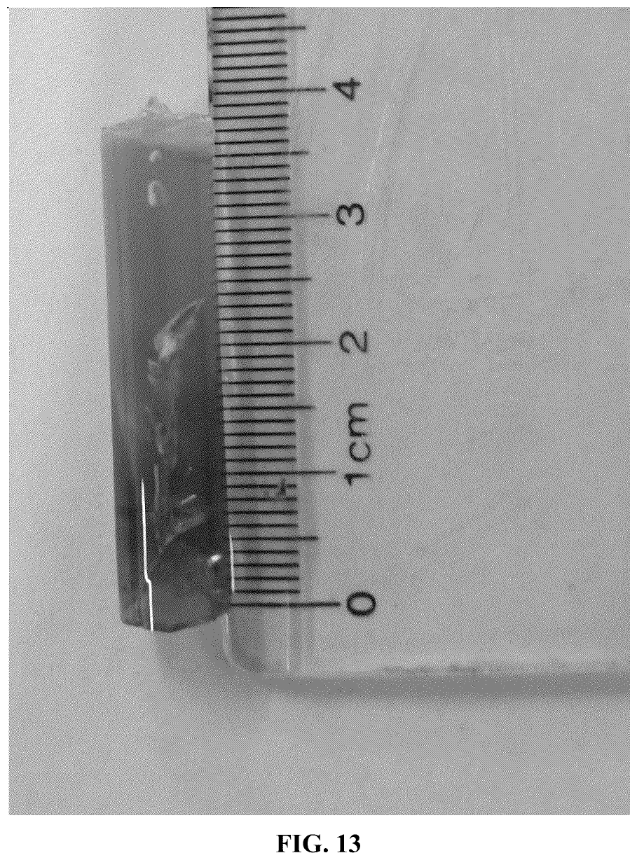

The macrograph of the deep-cured polymer prepared in Use Example 11 is shown in FIG. 13. As can be seen from FIG. 13, when the photocuring time is 30 min, a polymer curing depth of 3.8 cm is observed. It is thus demonstrated that the carbazole-based bis-chalcone can be used in combination with an iodonium salt as a photoinitiator for the preparation of deep-cured materials, with a wider application range.

In summary, in the present disclosure, the carbazole-based bis-chalcone can act as a photoinitiator to initiate a polymerization reaction of acrylate monomers, which shows a rapid photoinitiation rate and a high double-bond conversion and can be used in deep-curing systems.

The descriptions above are merely the preferred embodiments of the present disclosure. It should be noted that several improvements and modifications may also be made by those of ordinary skill in the art without departing from the principle of the present disclosure, and these improvements and modifications shall also be considered within the scope of the present disclosure.

What is claimed is:

1. A carbazole-based bis-chalcone having a chemical structural formula of Formula I: 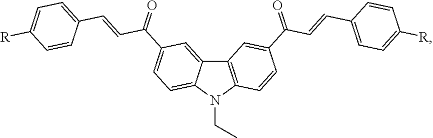
wherein in the Formula I, R is selected from the group consisting of —H, —SMe, —OMe, NMe₂, NEt₂, CF₃, and —N(Ph)₂.

2. A method for preparing the carbazole-based bis-chalcone of claim 1, comprising:
mixing 3,6-diacetyl-N-ethylcarbazole, an alkali, a solvent, and benzaldehyde or a benzaldehyde derivative, and subjecting a resulting mixture to a Claisen-Schmidt reaction to obtain the carbazole-based bis-chalcone.

3. The method of claim 2, wherein a molar ratio of the benzaldehyde or the benzaldehyde derivative to the 3,6-diacetyl-N-ethylcarbazole is in a range of 2:1 to 3:1.

4. The method of claim 2, wherein a ratio of an amount of substance of the 3,6-diacetyl-N-ethylcarbazole to a volume of the solvent is in a range of 5 mmol:30-80 mL.

5. The method of claim 2, wherein the Claisen-Schmidt reaction is conducted at a temperature of 30° C. to 80° C. for 8 hours to 15 hours.

6. A photoinitiator comprising the carbazole-based bis-chalcone of claim 1 and ethyl p-dimethylaminobenzoate or an iodonium salt.

7. A process for initiating a polymerization of an acrylate monomer, comprising using the carbazole-based bis-chalcone of claim 1.

8. The process of claim 7, wherein the acrylate monomer comprises at least one selected from the group consisting of methyl methacrylate, butyl methacrylate, methyl acrylate, n-butyl acrylate, 1,6-hexanediol diacrylate, trimethylolpropane triacrylate, pentaerythritol triacrylate, 2-hydroxypropyl methacrylate or 2-hydroxyethyl acrylate, 2,2-bis[4-(2-hydroxy-3-methacryloyloxypropoxy)phenyl]propane, triethylene glycol dimethacrylate, 3,3,4,4,5,5,6,6,7,7,8,8,8-tridecafluorooctyl acrylate, 2,2,3,3,4,4,5,5,6,6,7,7,8,8,8-pentadecafluorooctyl acrylate, hexafluorobutyl acrylate, pentafluorophenyl methacrylate, 2,2,2-trifluoroethyl acrylate, 2-(perfluorooctyl)ethyl methacrylate, 2-(perfluorobutyl)ethyl methacrylate, 2-(perfluorohexyl)ethyl acrylate, 2-(perfluorohexyl)ethyl methacrylate, 2,2,3,3-tetrafluoropropyl methacrylate, 2,2,3,3,3-pentafluoropropyl acrylate, 1H,1H,2H,2H-perfluorodecyl acrylate, 1H,2H,3H,4H-perfluorooctanol acrylate, 2,2,3,3,4,4,4-heptafluorobutyl acrylate, 2,2,3,3,4,4,4-heptafluorobutyl methacrylate, 2,2,3,4,4,4-hexafluorobutyl methacrylate, 1,1,1,3,3,3-hexafluoropropan-2-yl acrylate and 2,2,3,3,4,4,5,5-octafluoro-1,6-hexanediol diacrylate.

9. The process of claim 7, wherein a light source for the polymerization is an LED (light-emitting diode) light source.

10. A process for initiating a polymerization of an acrylate monomer, comprising using the photoinitiator of claim 6.

11. The process of claim 10, wherein the acrylate monomer comprises at least one selected from the group consisting of methyl methacrylate, butyl methacrylate, methyl acrylate, n-butyl acrylate, 1,6-hexanediol diacrylate, trimethylolpropane triacrylate, pentaerythritol triacrylate, 2-hydroxypropyl methacrylate or 2-hydroxyethyl acrylate, 2,2-bis[4-(2-hydroxy-3-methacryloyloxypropoxy)phenyl]propane, triethylene glycol dimethacrylate, 3,3,4,4,5,5,6,6,7,7,8,8,8-tridecafluorooctyl acrylate, 2,2,3,3,4,4,5,5,6,6,7,7,8,8,8-pentadecafluorooctyl acrylate, hexafluorobutyl acrylate, pentafluorophenyl methacrylate, 2,2,2-trifluoroethyl acrylate, 2-(perfluorooctyl)ethyl methacrylate, 2-(perfluorobutyl)ethyl methacrylate, 2-(perfluorohexyl)ethyl acrylate, 2-(perfluorohexyl)ethyl methacrylate, 2,2,3,3-tetrafluoropropyl methacrylate, 2,2,3,3,3-pentafluoropropyl acrylate, 1H,1H,2H,2H-perfluorodecyl acrylate, 1H,2H,3H,4H-perfluorooctanol acrylate, 2,2,3,3,4,4,4-heptafluorobutyl acrylate, 2,2,3,3,4,4,4-heptafluorobutyl methacrylate, 2,2,3,4,4,4-hexafluorobutyl methacrylate, 1,1,1,3,3,3-hexafluoropropan-2-yl acrylate and 2,2,3,3,4,4,5,5-octafluoro-1,6-hexanediol diacrylate.

12. The process of claim 10, wherein a light source for the polymerization is an LED light source.
